(19) United States
(12) Patent Application Publication
(10) Pub. No.: US 2002/0198392 A1
(43) Pub. Date: Dec. 26, 2002

(54) Leather finished with scent-containing microcapsules

(76) Inventors: Martin Kleban, Leverkusen (DE); Jurgen Weisser, Rommerskirchen (DE); Friedrich Koch, Koln (DE); Wolfgang Schwaiger, Leichlingen (DE)

Correspondence Address: BAYER CORPORATION, PATENT DEPARTMENT, 100 BAYER ROAD, PITTSBURGH, PA, 15205, US

(21) Appl. No.: 10/115,822

(22) Filed: Apr. 3, 2002

(30) Foreign Application Priority Data
Apr. 9, 2001 (DE) 10117671.6

(51) Int. Cl. A61K 38/00; C07C 229/00

(52) U.S. Cl. 554/104; 508/411; 564/238; 514/20

(57) ABSTRACT

This invention relates to a leather that contains scent-containing microcapsules in its cross section, wherein the walls of the microcapsules comprise reaction products of guanidine compounds and polyisocyanates.

### BACKGROUND OF THE INVENTION

The invention relates to leather finished with scent-containing microcapsules, to processes for production thereof, and also to specific cationized microcapsules, a process for their production, and their use.

State of the art processes for producing leather from hides and skins utilize a multiplicity of different natural and synthetic tanning, auxiliary, and finishing materials. The intrinsic odor of many of these products and of impurities they contain, particularly organic solvents, mean that the ready-produced leather has an odor which the consumer will only rarely equate with a pleasant, typical leather aroma.

The attempt to remedy this problem with simple, commercially available scent solutions is unsuccessful because of two problems:

1) Application of such preparations using processes and apparatus typical of leather is associated with substantial odor nuisance for employees.

2) The effect of such a preparation is only very shortlived, whereas leather is a very longlived product.

The use of microcapsule preparations for finishing leather is known and has been described for some examples.

DE-A 3,921,145, for example, describes a process for delustering leather surfaces by application of microcapsule-containing finishing binders. Here, all that matters for efficacy is the particle size, not the constitution of the particles or even their contents. Moreover, the microcapsules are applied with the binder purely surficially.

WO-A 00/65,100 discloses a process for finishing leather with microcapsules. In the disclosed process, the leather is compressed using a roller and, as it re-expands, absorbs the microcapsules. The chemical constitution of the capsules is not further specified.

### SUMMARY OF THE INVENTION

There has now been found a leather containing scent-containing microcapsules in its cross section, wherein the walls of the microcapsules comprise reaction products of guanidine compounds and polyisocyanates.

### DETAILED DESCRIPTION OF THE INVENTION

The leather according to the invention preferably contains scent-containing microcapsules in 50% (preferably in 80%) of its cross section. Preferably the average particle size of the microcapsules is 2 to 20 μm. The content of scent-containing microcapsules in the leather is preferably 0.1 to 10% by weight (especially 0.5 to 3% by weight), based on the weight of the finished leather.

Useful scents include all commercially available hydrophobic and hence water-insoluble scents as described, for example, by P. Kraft et al. in Angew. Chem., 112, 3106-3138 (2000). In the case of substances that are soluble in water as well as in oils, the addition of odor-neutral, sparingly volatile oils such as paraffins, alkylaromatics, or esters can enable use.

The retention properties of the capsules can be influenced in a very simple manner by varying the wall thickness. This makes it possible to create slow release capsules that, when applied to the leather, continuously release scent for a prolonged period, or even virtually odorless leathers that release scent only under mechanical pressure.

Preferred wall thicknesses for the scent-containing microcapsules are in the range of 2 to 25% (preferably 3 to 15% and especially 4 to 10%) of wall fraction, each percentage being based on the sum total of the capsule ingredients. The wall fraction of the microcapsules is directly proportional to the fraction of the primary wall-former, the polyisocyanate.

Useful guanidines for producing the microcapsules include, for example, those of the formula (I)

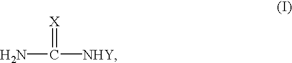

or their salts with acids,

where

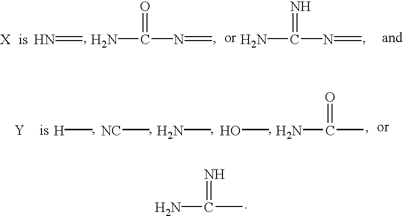

Useful salts include, for example, the salts of carbonic acid, nitric acid, sulfuric acid, hydrochloric acid, silicic acid, phosphoric acid, formic acid, and/or acetic acid. The use of salts of guanidine compounds of the formula (I) can take place in combination with inorganic bases in order to obtain the free guanidine compounds of the formula (I) from the salts in situ. Useful inorganic bases for this purpose include, for example, alkali and/or alkaline earth metal hydroxides and/or alkaline earth metal oxides. Preference is given to aqueous solutions or slurries of these bases, especially aqueous sodium hydroxide solution, aqueous potassium hydroxide solution, and aqueous solutions or slurries of calcium hydroxide. It is also possible to use combinations of two or more bases.

It is often advantageous to use the guanidine compounds of the formula (I) as salts, since they are commercially available in that form and free guanidine compounds are in some instances sparingly soluble in water or lack stability in storage. When inorganic bases are used, they may be used in stoichiometric, substoichiometric, or superstoichiometric amounts, based on salts of guanidine compounds. Preference is given to using 10 to 100 equivalent-% of inorganic base, based on salts of the guanidine compounds. The addition of inorganic bases has the consequence that, for microencapsulation, guanidine compounds having free NH₂ groups are available in the aqueous phase for reaction with the polyisocyanates in the oil phase. For microencapsulation, salts of guanidine compounds and bases are advantageously added separately to the aqueous phase.

Preference is given to using guanidine or salts of guanidine with carbonic acid, nitric acid, sulfuric acid, hydrochloric acid, silicic acid, phosphoric acid, formic acid, and/or acetic acid.

It is particularly advantageous to use salts of guanidine compounds with weak acids. These are in equilibrium with the corresponding free guanidine compound in aqueous solution as a consequence of hydrolysis. The free guanidine compound is consumed during the encapsulation process and is constantly regenerated according to the law of mass action. Guanidine carbonate exhibits this advantage to a particular degree. When salts of guanidine compounds with weak acids are used, there is no need to add inorganic bases to release the free guanidine compounds.

Useful guanidine compounds of the formula (I) for the present invention may also be prepared by ion exchange from their water-soluble salts according to the prior art using commercially available basic ion exchangers. The eluate from the ion exchanger can be neutralized directly for capsule wall formation by mixing it with the oil-in-water emulsion.

For example, sufficient guanidine compound can be used so that 0.2 to 4.0 mol of free NH₂ groups are introduced into or released in the water phase in the form of guanidine compounds per mole of NCO groups present as polyisocyanate in the oil phase. This amount is preferably 0.5 to 1.5 mol. When guanidine compounds are used in a substoichiometric amount, free NCO groups remain after the reaction with the polyisocyanate. These then generally react with water, which is usually not critical since this reaction gives rise to new free amino groups capable of crosslinking.

The guanidine compounds are preferably used in the form of aqueous solutions. The concentration of such solutions is not critical and is generally limited only by the solubility of the guanidine compounds in water. Useful aqueous solutions of guanidine compounds are 1 to 20% by weight in strength, for example.

Useful polyisocyanates for producing the scent-containing microcapsules according to this invention include a very wide range of aliphatic, aromatic, and aromatic-aliphatic difunctional and higher functionality isocyanates, especially those known for producing microcapsules. Preference is given to using aliphatic polyisocyanates. Particular preference is given to using hexamethylene diisocyanate, isophorone diisocyanate, and/or derivatives of hexamethylene diisocyanate and of isophorone diisocyanate that have free isocyanate groups and contain biuret, isocyanurate, uretidione, and/or oxadiazinetrione structures. Mixtures of different polyisocyanates can also be used. Some useful polyisocyanates are described for example in EP-A 227,562, EP-A 164,666, and EP-A 16,378.

In a preferred embodiment of the leather according to the invention, the microcapsules have walls comprising reaction products of guanidine compounds, polyamines, and polyisocyanates.

The guanidine compound is preferably used in an amount of 0.5 to 0.99 mol equivalents (especially 0.51 to 0.75 mol equivalents), based on polyisocyanate, and the polyamine compound is used in an amount of 0.1 to 1 mol equivalents (especially 0.5 to 0.75 mol equivalents), based on polyisocyanate, the total amount of guanidine compound and polyamine being greater than 1.1 mol equivalents, based on polyisocyanate.

Such capsules are cationizable, for example, by acid action or alkylation, to produce microcapsules whose use is likewise preferred.

The invention therefore also provides, for example, acid- or alkylation-cationized microcapsules and also their cationizable precursors for which the walls comprise reaction products of guanidine compounds, polyamines, and polyisocyanates.

Useful polyfunctional amines include primarily linear and/or branched polyalkyleneamines having a molecular weight less than 5000, but are preferably readily water-soluble polyethyleneamines such as diethylenetriamine, triethylenetetramine, tetraethylenepentamine, pentaethylenehexamine, or triaminoethyleneamine and also mixtures thereof. Permanent cationization can be effected not only before but also after capsule formation using customary alkylating reagents, for example, dimethyl sulfate.

Preference is likewise given to the leather according to the invention containing preferably alkylation-cationized microcapsules for which the walls comprise reaction products of guanidine compounds and polyisocyanates.

The invention therefore also provides corresponding cationized microcapsules and also the corresponding cationizable microcapsules.

The cationized or cationizable microcapsules according to the invention are capable, especially in an application process from an aqueous float, to absorb effectively and substantively onto leather. This provides good effects even with low use levels even in a float process.

The microcapsules according to the invention may, as will be appreciated, contain other encapsulated compounds as well, for example, dye precursors, adhesives, pharmaceuticals, insecticides, fungicides, herbicides, and repellents. The microcapsules according to the invention can be applied not just to leather but also to paper and textile and other substrates.

The invention further provides aqueous dispersions of the microcapsules according to the invention. The dispersions according to the invention preferably contain 5 to 60% by volume (especially 25 to 52% by volume) of microcapsules, based on the aqueous dispersion.

The invention further provides a process for producing the leather finished according to the invention in which the scent-containing microcapsules, preferably an aqueous dispersion of microcapsules, are applied to the flesh side of the leather, preferably by spraying or film or roller coating, or are applied to the leather by the exhaust process.

The leather finishing process according to the invention is preferably effected using 0.1 to 10% by weight (especially 0.5 to 3% by weight) of microcapsules.

In a preferred embodiment of the process, application is effected in the float by means of exhaust processes using especially the cationized or cationizable microcapsules according to the invention. Exhaustion from the float preferably amounts to more than 75% and especially to more than 90%.

The microcapsules according to the invention are preferably added to the tanning float during the retanning operation or during the concluding fatliquoring operation.

The production of leather and furs from hides and skins commonly takes place in more than one operation. Following the preparatory operations in the beamhouse, such as dehairing, defleshing, deliming, and bating, a typical processing sequence consists of tanning, retanning, dyeing, fatliquoring, and finishing. The individual operations may be divided into further subsidiary units.

While tanning leads to an increase in the shrinkage temperature of the leather, retanning has virtually no effect in that regard. By “retanning” is meant the aftertreatment of pretanned (generally chrome-tanned) leather in order to optimize color, levelness, softness, fullness, and hydrophobicity and to fix tanning materials.

The microcapsules are added in a pH of 3 to 6 and especially 4.5 to 5.9, preferably as an aqueous dispersion. Preferably the microcapsules are allowed to penetrate into the leather before—and this is likewise a preferred variant of the process—they are fixed in the leather by setting a pH of 3 to 4.5 and preferably 3.4 to 4. Fixation is advantageous especially in the case of the microcapsules according to the invention that are cationized latently, i.e., by acid action.

Examples of organic water-immiscible and inert solvents that, together with the material to be encapsulated and the polyisocyanate, form part of the oil phase during the production of the microcapsules according to the invention and used according to the invention include aromatic, aliphatic, and naphthenic hydrocarbons, carboxylic esters, chlorinated paraffins, oils of animal and vegetable origin, natural fats having melting points in the range from 10° C. to 35° C., and aromatic and aliphatic ethers boiling above 100° C. Mixtures of a plurality of solvents can also be used.

The scent-containing microcapsules may be produced using the aqueous phase optionally containing emulsifiers, stabilizers and/or anti-coalescers. Emulsifiers may also be present in the oil phase, if desired. The amount of such additives may be for example in the range from 0 to 2% by weight, based on the respective phase.

It will be appreciated that the scents to be encapsulated must not react with isocyanates under the encapsulation conditions.

The scent-containing microcapsules can be produced by conventional continuous and batchwise processes, and crosslinkers then to be used are not the customary polyamines but are guanidine compounds. More particularly, guanidine compounds of the formula (I) or their salts are used, optionally in combination with inorganic bases. Similarly, the use of basic salts of guanidine compounds with weak acid leads to good results. This approach is industrially particularly advantageous, since the free base does not have to be prepared separately, for example, by addition of inorganic bases or by ion exchange.

Similarly, the cationized microcapsules according to the invention can be produced as described above. Cationization is effected subsequently, preferably by acid action or alkylation.

When the microcapsules according to the invention are produced on the basis of guanidine compounds, optionally polyamines and polyisocyanates, the polyamine is preferably not used until after the polyisocyanate has been crosslinked with the guanidine compound.

Both the production of the emulsion containing droplets of an oil phase and a continuous aqueous phase and the addition of guanidine compounds can be carried out continuously and batchwise.

An example of a batchwise operation has an emulsion that contains oil droplets approximately in the size of the desired microcapsules having added to it at 10 to 50° C. a sufficient amount of a guanidine compound as is stoichiometrically required for the reaction of all isocyanate groups present in the oil phase. When guanidine compounds are available in the form of salts, it is optionally possible to use an anion exchanger to first recover an aqueous solution of the free guanidine compound from an aqueous solution of the particular salt and to use this aqueous solution of a free guanidine compound. It is assumed that all NH₂ groups present in guanidine compounds or formed from salts of guanidine compounds can react with NCO groups. In the case of guanidine and guanidine compound salts (formula (I), where X=NH and Y=H) it is thus assumed that one mole thereof can react with 2 mol of NCO groups.

The meeting between free guanidine compounds and polyisocyanates present in the oil phase is responsible for the start of a polyaddition reaction that is also known as crosslinking at interfaces of the oil droplets with the aqueous phase. This polyaddition or crosslinking reaction can be completed if desired at an elevated temperature, for example, up to the boiling point of the aqueous phase. The result is a dispersion of microcapsules in water for which the capsule content can be up to about 60% by weight. Capsule content here is the weight ratio of oil phase inclusive of isocyanate to the aqueous phase in the starting emulsion. The calculation of the capsule content does not take account of the guanidine compound involved in wall formation and any inorganic base used.

The above-mentioned emulsion may also have salts of guanidine compounds added to it. In that case, the temperature is maintained below 60° C. and an inorganic base of the kind described can then be added, preferably in a stoichiometric amount, based on the salt. In the process, guanidine compounds are released in situ and then react in the above-mentioned manner. In the case of salts of guanidine compounds and weak acids, which are hydrolytically cleaved in the presence of water and then contain fractions of free guanidine compounds, the addition of inorganic bases can be omitted. This applies to guanidine carbonate in particular.

A continuous operation can be carried out, for example, by generating an emulsion of the desired type and oil droplet size continuously by flow through an emulsifying machine. This can be followed by the continuous addition, for example, at 25 to 50° C., without the action of shearing forces, of an aqueous solution of a guanidine compound and, if appropriate, in a downstream reaction vessel, if necessary, the inorganic base required to release guanidine compounds from salts. The polyaddition reaction can then be completed in further reaction vessels, if necessary at temperatures up to 100° C.

The present invention also provides a process for producing the microcapsule dispersions according to the invention by emulsifying an oil phase containing an organic water-immiscible inert solvent, the scent to be encapsulated, and a polyisocyanate in a water phase optionally containing additives and adding to the emulsion a guanidine compound capable of entering addition reactions with isocyanate groups.

The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

### Example 1

### Capsules Filled with Scent

While cooling, 0.7 liter of a 0.8% solution of polyvinyl alcohol 26/88 (Airvol® 523, Air Products) in water was initially charged and 0.3 liter of a solution of 21 g of polyisocyanate (HDI biuret, NCO content about 22%) in 300 ml of scent was added in the course of 40 sec with stirring. This was followed by a further 4 min of emulsification using a high-speed rotor-stator mixer (temperature 20 to 25° C.) to obtain the desired average particle size. 53 g of 10% guanidinium carbonate solution were then added and the dispersion was gradually heated to 70° C. (2 h) with stirring. After a further 2 h at 70° C., the dispersion was cooled to room temperature and stabilized by addition of 40 ml of thickener (modified starch).

### Example 2

### Capsules Filled with Scent and Neutral Oil

While cooling, 0.5 liter of a 1.2% solution of polyvinyl alcohol 26/88 (Airvol® 523, Air Products) in water was initially charged and 0.5 liter of a solution of 35 g of polyisocyanate (HDI biuret, NCO content about 23%) in 50 ml of scent and 450 ml of diisopropyinaphthalene was added in the course of 40 sec with stirring. This is followed by a further 4 min of emulsification using a high-speed rotor-stator mixer (temperature 20 to 25° C.) to obtain the desired average particle size. 88 g of 10% guanidinium carbonate solution were then added and the dispersion was gradually heated to 70° C. (2 h) with stirring. After a further 2 h at 70° C., the dispersion was cooled to RT and stabilized by addition of 40 ml of thickener (modified starch).

Appearance and storage stability of capsule dispersions of examples 1 and 2:

| | | | Particle size | |
|---|---|---|---|---|
| N° | Scent | Isocyanate | [μm]; distribution | Slurry |
| 1a | Blue Line | HDI biuret | 7.2; 2.1 | white |
| 1b | Cuir Naturell | HDI biuret | 6.9; 2.0 | white |
| 1c | Blue Line | HDI trimer | 7.0; 2.2 | white |
| 1d | Blue Line | HDI biuret + | 11.5; 1.6 | white |
| | | PMDI 1:1 | | |
| 2a | Blue Line | HDI biuret | 6.0; 1.3 | white |
| 2b | Lennox | HDI biuret | 5.7; 1.3 | white |
| 2c | Cuir Naturell | HDI biuret | 5.8; 1.4 | white |
| 2d | Frutti di Bosco | HDI biuret | 3.1; 1.8 | white |
| 2e | Ozonodor | HDI biuret | 4.0; 1.4 | white |

Scents: Products from Haarmann & Reimer, Holzminden:

Blue Line: mixture of methylisopropylcyclohexene, (diisopropylphenyl)-methylpropanal, lemon oil, and dimethyloctadienol in diethyl benzenedicarboxylate

Cuir Naturell: mixture of dimethylphenol, benzyl alcohol, phenylethyl alcohol, cresol, benzyl benzoate, and terpineol in diethyl benzenedicarboxylate

Frutti di Bosco: mixture of benzyl benzoate, benzyl alcohol, benzaldehyde, allyl caproate, methyl salicylate, orange oil, and clove flower oil

Ozonodor: mixture of turpentine oil, pine needle oil, and eucalyptus oil in trimethylbicycloheptanyl acetate

HDI biuret: NCO content about 23%, viscosity about 2500 mPas

HDI trimer: NCO content about 22%, viscosity about 3500 mPas

PMDI: NCO content about 32%, viscosity about 3000 mPas

HDI=hexamethylene diisocyanate

PMDI=polymethylene diphenyl diisocyanate

Particle Size:

Ø: average diameter in μm (by volume); distribution: broad distribution (d₉₀−d₁₀)/d₅₀ (by volume)

### Example 3

### Finishing of Leather by Add-On Process

The capsule slurry of Example 1a was 1:10 diluted with water and applied to a commercially available furniture leather (cattlehide, 1.5 mm, chrome tanned, aniline type) on the flesh side by:

a) spraying with 2 times about 100 ml/m², intermediate and supplementary drying 1 min at 80° C.

b) film coater, add-on about 200 ml/m², drying 1 min at 80° C.

After drying, no significant changes in leather properties (hand, softness) were observed. The leather obtained had a pleasant smell that increases on mechanical exposure/agitation. The effect was still noticeable several weeks later.

When the capsule preparation of Example 2a was instead used, the leather obtained was virtually odorless after drying, but the desired effect appeared on or after mechanical loading.

### Example 4

### Finishing of Leather by Add-On Process with a Dye Capsule

To demonstrate the penetration of the leather, a capsule was prepared similarly to Example 2 except that a solution of 4% of crystal violet lactone in diisopropylnaphthalene was used as capsule content. Crystal violet lactone is colorless under neutral conditions but turns a deep blue-violet on contact with moisture and acid (as present in the leather).

Crystal violet lactone was applied as in Example 3a by spraying.

Surface and cross section of the finished leather were colorless, which means that no free dye was present and proves that the microcapsules were not damaged by the process of application.

The capsules could be destroyed by vigorous agitation of the leather or rubbing on the cut surface of the leather. Then released dye colored the cross section of the leather a uniform blue, proving the uniform distribution of the capsules throughout the entire leather cross section.

### Example 5

### Latently Cationic Capsule

While cooling, 0.5 liter of a 0.8% solution of polyvinyl alcohol 26/88 (Airvol® 523, Air Products) in water was initially charged and 0.3 liter of a solution of 21 g of polyisocyanate (HDI biuret, NCO content about 23%) in 300 ml of “Blue Line” scent was added in the course of 40 sec with stirring. This was followed by a further 4 min of emulsification using a high-speed rotor-stator mixer (temperature 20 to 25° C.) to obtain the desired average particle size. 40 g of 10% guanidinium carbonate solution were then added and the dispersion was gradually heated to 70° C. (2 h) with stirring. 50 ml of a 10% solution of pentaethylenehexamine in water were then added. After a further 2 h at 70° C., the dispersion was cooled to room temperature and stabilized by addition of 40 ml of thickener (modified starch).

### Example 6

### Latently Cationic Capsule

While cooling, 0.5 liter of a 1.2% solution of polyvinyl alcohol 26/88 (Airvol® 523, Air Products) in water was initially charged and 0.5 liter of a solution of 35 g of polyisocyanate (HDI biuret, NCO content about 23%) in 50 ml of “Blue Line” scent and 450 ml of diisopropyinaphthalene was added in the course of 40 sec with stirring. This was followed by a further 4 min of emulsification using a high-speed rotor-stator mixer (temperature 20 to 25° C.) to obtain the desired average particle size. 60 g of 10% guanidinium carbonate solution were then added and the dispersion was gradually heated to 70° C. (2 h) with stirring. 70 ml of a 10% solution of pentaethylenehexamine in water were then added. After a further 2 h at 70° C., the dispersion was cooled to room temperature and stabilized by addition of 40 ml of thickener (modified starch).

### Example 7

### Permanently Cationic Capsule

7 g of dimethyl sulfate were added to a capsule dispersion prepared as in Example 5, before thickening, and the mixture was heated to 50° C. After 2 h of stirring at 50° C., the dispersion was cooled to room temperature and stabilized by addition of thickener.

### Example 8

### Finishing of Leather in the Drum, Float Process

Raw material: wet blue, cattlehide, 2 mm; percentages based on shaved weight of wet blue

Procedure:

The wet blue was combined with the retanning materials and fats at about pH 5. Microcapsules could be added (addition time A). Retanning materials, microcapsules (if used) and fats then penetrated the leather before they were fixed at pH 3.5 to 4.

The microcapsules could also be added during the subsequent final fatliquoring (addition time B).

An illustrative addition sequence of ingredients demonstrates this below:

| | | | Time | |
|---|---|---|---|---|
| | % | Product | min | Remarks |
| | 200 | Water 40° C. | | |
| | 0.2 | Formic acid 1:10 | 15 | pH: 3.4 |
| | | | | Float dropped |
| | 100 | Water 40° C. | | |
| | 1.0 | Dye 1:20 | 20 | |
| + | 2.0 | Neutral salt, (aromatic sulfonic acids) | | |
| | 2.0 | Syntan (condensate of aromatic | | |
| | | sulfonic acids) | | |
| | 1.5 | Sodium formate | 30 | pH: 4.2 |
| + | 0.5 | Sodium bicarbonate | 30 | pH: 4.8 |
| | | | | Float dropped |
| | 300 | Water 40° C. | 10 | Float dropped |
| | 50 | Water 40° C. | | |
| | 3.0 | Softening polytan | 20 | pH: 5.2 |
| + | 6.0 | Syntan (condensate of aromatic | | |
| | | sulfonic acids) | | |
| | 3.0 | Resin tanning material | 30 | |
| + | 2.0 | Dyeing auxiliary (aromatic sulfonic | | |
| | | acids) | | |
| | 3.0 | Dye | | |

| | | | Time | |
|---|---|---|---|---|
| | % | Product | min | Remarks |
| | 3.0 | Test product, addition time A | 60 | |
| + | 50 | Water 50° C. | 5 | |
| + | 0.5 | Formic acid 1:10 | 15 | |
| + | 0.5 | Formicacid 1:10 | 30 | pH: 3.9 |
| | | | | Float dropped |
| | 50 | Water 50° C. | | |
| | 0.5 | Ammonia 1:10 | 5 | |
| + | 2.0 | Test product, addition time B | | |
| | 8.0 | Synthetic fatliquor 1:4 | 60 | |
| + | 1.0 | Formic acid 1:10 | 30 | pH: 3.8 |
| | | | | Float dropped |
| | 300 | Water 40° C. | 10 | Float dropped |

Leather onto horse, set out, vacuum dried at 60° C. for 1½ min, hung up, fully dried, and finished in conventional manner.

| | | Addition | Float | |
|---|---|---|---|---|
| Ex. | Test product | time | exhaustion | Remarks |
| 8a | “Blue Line” | B | Not | substantial odor nuisance |
| | scent | | determinable | during tanning, odor in |
| | | | | leather disappeared within |
| | | | | a few days |
| 8b | Capsules of | A | 90% | leather had persistent |
| | Example 4 | | | unpleasant odor, odor |
| | | | | increased by agitation |
| 8c | Capsules of | B | 77% | leather had persistent |
| | Example 4 | | | unpleasant odor, odor |
| | | | | increased by agitation |
| 8d | Capsules of | A | 91% | almost odorless leather |
| | Example 5 | | | gave off pleasant smell on |
| | | | | agitation |
| 8e | Capsules of | B | 94% | almost odorless leather |
| | Example 6 | | | gave off pleasant smell on |
| | | | | agitation |

What is claimed is:

1. A leather containing scent-containing microcapsules in its cross section, wherein the walls of the microcapsules comprise reaction products of guanidine compounds and polyisocyanates.

2. A leather according to claim 1 wherein the content of scent-containing microcapsules in the leather is 0.1 to 10% by weight, based on the weight of the finished leather.

3. A process for producing a finished leather according to claim 1 comprising applying the scent-containing microcapsules to the flesh side of the leather or to the leather by the exhaust process.

4. A process according to claim 3 wherein the microcapsules are cationized or acid-cationizable and are applied as a float using an exhaust process.

5. Alkylation-cationized or acid-cationized microcapsules having walls comprising reaction products of guanidine compounds, polyamines, and polyisocyanates.

6. Cationizable microcapsules having walls comprising reaction products of guanidine compounds, polyamines, and polyisocyanates.

7. An aqueous dispersion containing cationized microcapsules according to claim 5.

8. An aqueous dispersion containing cationizable microcapsules according to claim 6.

9. A method comprising finishing leather with alkylation-cationized or acid-cationized microcapsules according to claim 5 that contain encapsulated ingredients.

10. A method according to claim 9 wherein an encapsulated ingredient is a scent.

11. A method comprising finishing leather with cationizable microcapsules according to claim 6 that contain encapsulated ingredients.

12. A method according to claim 11 wherein an encapsulated ingredient is a scent.
